(19) United States
(12) Patent Application Publication
(10) Pub. No.: US 2009/0191274 A1
(43) Pub. Date: Jul. 30, 2009

(54) Pharmaceutical composition of amlodipine maleate having enhanced stability

(76) Inventors: Sang-Joon Lee, Gunpo-si (KR); Hee-Jong Shin, Bucheon-si (KR); Jong-Lae Lim, Cheonan-si (KR); Seok-Kyu Lee, Cheonan-si (KR); Soo-Yeon Moon, Cheonan-si (KR); Shin-Jung Park, Cheonan-si (KR); Taek-Hwan Shin, Cheonan-si (KR)

Correspondence Address: SUGHRUE MION, PLLC, 2100 PENNSYLVANIA AVENUE, N.W., SUITE 800, WASHINGTON, DC, 20037, US

(21) Appl. No.: 10/585,631

(22) PCT Filed: Jan. 14, 2005
(86) PCT No.: PCT/KR05/00120

§ 371 Date: Jul. 11, 2006

(30) Foreign Application Priority Data
Jan. 16, 2004 (KR) 10-2004-0003439

(51) Int. Cl. A61K 9/12; A61K 31/4422

(52) U.S. Cl. 424/490; 514/356; 424/494; 424/497

(57) ABSTRACT

The present invention relates to a coated particle of amlodipine maleate and a pharmaceutical composition for cardiovascular disease (CVD) comprising the coated particle of amlodipine maleate. The pharmaceutical composition of the present invention has stable bioavailability due to sufficient dissolution rate and prevents decomposition reaction of amlodipine to thereby being formulated with formulation-stability equal to or higher than amlodipine besylate under long storage.

### TECHNICAL FIELD

The present invention relates to formulation-stability of amlodipine maleate. The present invention may provide a coated particle of amlodipine maleate and pharmaceutical composition thereof with formulation-stability equal to or higher than amlodipine besylate regardless of pH of composition during long storage due to coating a particle of amlodipine maleate with pharmaceutically acceptable coating agent and preventing decomposition reaction of amlodipine maleate.

### BACKGROUND ART

It is known that decomposition reaction of drug is complexly affected by pH, water or crystalline phase, purity or particle size of drug and the like.

Amlodipine is useful compound as calcium channel blocker for treating a wide variety of cardiac conditions, particularly cardiovascular disease (CVD) including angina, hypertension, etc.

European Patent Laid Open Publication Number 89,167 has disclosed various forms of pharmaceutically acceptable salts of amlodipine, particularly pharmaceutically acceptable acid addition salts, such as hydrochloride, hydrobromide, sulfate, phosphate or acid phosphate, acetate, maleate, fumarate, lactate, tartrate, citrate or gluconate, preferably maleate.

However, formulations comprising amlodipine maleate are not commercially developed due to poor stability by decomposition reaction during storage.

Korean Patent Laid-Open Publication Number 1995-7228 has disclosed amlodipine besylate that may overcome problems related with formulation-stability of amlodipine maleate.

In the prior art, stability on besylate, mesylate, succinate, salicylate, maleate, tosylate, acetate and hydrochloride of amlodipine was evaluated using a mixture, as vehicle of tablet, of microcrystalline cellulose of 50:50 and anhydrous dibasic calcium phosphate, and as a result, it is disclosed that amlodipine besylate is most stable.

Drug comprising amlodipine has been approved as amlodipine besylate. Although Pitcher co., Ltd. initially had applied amlodipine maleate to United States Food and Drug Administration (US FDA), converted amlodipine maleate from amlodipine besylate when US FDA reviewed effective ingredient of the drug.

However, amlodipine besylate has problems that bioavailability of each individual represents in a large range of about 64 to about 90% due to high deviations of solubility and dissolution rate under pH conditions. And benzene sulfonate, with corrosive wear on skin or eyes and respiratory toxicity due to inhalation, is used during manufacture of amlodipine besylate, thereby to cause stability problem on workers and environmental toxicity during synthesis of drug.

To enhance formulation-stability of amlodipine, development of new salts of amlodipine has been tried. Korean Patent Laid-Open Publication Number 2002-76561 has disclosed amlodipine camsylate manufactured by reaction with amlodipine and camphorsulfonic acid, Korean Patent Laid-Open Publication Number 2003-81006 has disclosed amlodipine nicotinate, Korean Patent Laid-Open Publication Number 2004-11752 has disclosed tetrahydro-5-oxo-2-furane carboxylate of amlodipine, Korean Patent Laid-Open Publication Number 2003-17380 has disclosed a pyroglutamic acid salt of amlodipine, Korean Patent Laid-Open Publication Number 2004-72363 has disclosed a cyclamate of amlodipine. However, the new salts were developed related with the patent for amlodipine besylate and the try is weak to solve the problem for formulation-stability of amlodipine maleate evaluated the most preferred salts.

Further, Korean Patent Laid-Open Publication Number 2003-70594 has disclosed that a pharmaceutical composition, comprising amlodipine maleate, has excellent stability at a pH of about 5.5 to about 7.0, when measured as aqueous slurry of 20 weight percent.

Although the pharmaceutical composition comprising amlodipine maleate has efficiencies on stability due to selecting excipients so as to having of the pharmaceutical composition at a pH of about 5.5 to about 7.0, the stability of the pharmaceutical composition has equal to or less than one of amlodipine besylate according to test of stability described in Korean Patent Laid-Open Publication Number 2003-70594.

Korean Patent Publication Number 1995-7228 has disclosed that amlodipine maleate is unstable than amlodipine besylate, based on evaluation of stability of a pharmaceutical composition comprising amlodipine maleate and a pharmaceutical composition including amlodipine besylate when pH of the pharmaceutical compositions has a range of about 5.5 to about 7.0.

The present inventors has discovered that amlodipine maleate may be easily decomposed by direct contact with excipients during studying on amlodipine maleate with improving bioavailability due to high solubility. Accordingly, the present inventors prepared amlodipine maleate by coating to thereby preventing decomposition reaction of amlodipine maleate without direct contact of excipients and/or regardless pH of a pharmaceutical composition comprising amlodipine maleate.

### DISCLOSURE OF THE INVENTION

### Technical Problem

To overcome problems of amlodipine maleate, that has excellent bioavailability due to high solubility thereof but commercially not developed due to decomposition reaction of drugs containing amlodipine maleate, it is an object of the present invention to provide a pharmaceutical composition with formulation-stability, comprising amlodipine maleate, for treating cardiovascular disease (CVD).

### Technical Solution

To accomplish the above-mentioned object, the present invention provides a coated particle of amlodipine maleate.

The present invention also provides pharmaceutical composition, comprising the coated particle of amlodipine maleate as active substance and pharmaceutically acceptable carriers, for treating cardiovascular disease (CVD).

The pharmaceutical composition comprising the coated particle of amlodipine has excellent bioavailability and may sale commercially due to improving formulation-stability during long storage regardless pH of the pharmaceutical composition. Preventing decomposition reaction of amlodipine maleate and decreasing a loss of amlodipine equal to or more than a loss of amlodipine besylate cause improvement of formulation-stability.

Formulations containing amlodipine are prepared for rapidity and stability after oral dosing such that formulations need to have sufficient dissolution rate in vitro. Therefore the pharmaceutical composition comprising the coated particle of amlodipine maleate of the present invention further is necessary to be prepared for coating of amlodipine maleate without decreasing a dissolution rate in vitro.

The pharmaceutical composition comprises the coated particle with a dissolution rate of about 80 weight percent by a dissolution experiment, which is carried to using 0.01 mol/L hydrochloric acid aqueous solution of 500 mL for about 30 minutes at about 75 rpm by conventional methods.

The pharmaceutical composition comprising the coated particle of amlodipine maleate has excellent bioavailability as well as formulation-stability equal to or more than a pharmaceutical composition comprising amlodipine besylate.

In part of pharmacy, drugs is conventionally coated using coating agent for intercepting unsavory and a bad smell, enhancing a value of product by change of color, controlling dissolution amount of drugs, and protecting acid-laid drugs from acid in the stomach for oral-dosing and unstable drug from exterior circumstances such as water, light, etc. And the drugs are coated as a unit formulation, such as granule, tablet, etc.

Although a pharmaceutical composition comprising amlodipine maleate is coated as a unit formulation by a conventional coating method, amlodipine maleate is decomposed. In Korean Patent Laid-Open No. 2003-70594, is disclosed a pharmaceutical composition comprising amlodipine maleate and excipients selected properly in order to have a pH range of about 5.5 to about 7.0 of the above pharmaceutical composition, but the above pharmaceutical composition has without formulation-stability. Thus the present inventors have known that amlodipine maleate may be decomposed due to direct contacting with excipients.

The pharmaceutical composition comprises the coated particle of amlodipine maleate, as an active substance for prevent of the direct contact. The pharmaceutical composition of the present invention may contain one or a mixture of above two selected from talc, light silicic anhydride, silicon dioxide, etc. for stability of amlodipine maleate and prevent of electricity and moisture during preparation of the pharmaceutical composition. Further, the pharmaceutical composition may include pharmaceutical acceptable colors for value of production.

The present invention has efficiencies on formulation-stability regardless size of the coated particle such that the size of the particle is unlimited.

In the present invention, may be used coating agent for the coated particle of amlodipine maleate, such as a water-soluble polymer group, a saccharine group and a water-unsoluable polymer group that may be broken in 0.01 mol/L of hydrochloric acid solution.

The water-soluble polymer group may comprise one or a mixture of above two selected from polyvinylpyrolidone, a cellulose group (hydroxypropylmethylcellulose, hydroxypropylmethylcellulose phthalate, methylcellulose, sodium carboxymethylcellulose, hydroxyethylcellulose, hydroxypropylcellulose, etc.), pectin, galactomannan, gelatin, a polyethylenglycol group, polymethacrylate, a cyclodextrin group (betacyclodextrin, methylbetacyclodextrin, hydroxypropylbetacyclodextrin), carbomer, polyvinylalcohol, etc. Preferably, coating agents of OPADRY® (Colorcon Co., Ltd.) and Eudragit® (Rohm Co., Ltd.) are used for ease of coating and rapid dissolution rate.

The saccharine group, as the coating agent, may comprise sugar, sorbitol, mannitol, etc., which may be used one or a mixture of above two selected from the saccharines. With the coating agent, may be used a mixture of above one selected from excipients, diluents, surfactants, lubricants, antifoaming agents, plasticizer for ease of manufacture and obtainment of excellent coating.

An object of a method of the present invention is accomplished by coating a particle of amlodipine maleate using fluidized bed granulator. The method comprises flowing a particle of amlodipine maleate using fluidized bed granulator and dispersing the coating agent on the particle of amlodipine maleate to thereby forming the coated particle of amlodipine maleate.

In flowing of the method, amlodipine maleate may be flowed alone in fluidized bed granulator, and/or one or above two of talc, silicic anhydride, silicon dioxide, etc. may be put into the fluidized bed granulator, for stabilizing of amlodipine maleate and preventing electricity and moisture, with amlodipine maleate, flowed and coated.

Solid formulations, such as capsules, tablets, powders, granules, etc., including the coated particle of amlodipine maleate are prepared by conventional methods, and may contain, as occasion demands, above 2 or 1 respectively selected from excipients, sweeteners, flavors, diluents, lubricants, surfactants, thickening agents, pH control agents, stain, antifoaming agent etc. so as to dose into patients directly.

The solid formulation comprising the coated particle of amlodipine maleate is prepared by coating the particle of amlodipine maleate using fluidized bed granulator and formulizing as tablets, capsules, powders, etc. such that decomposition reaction of amlodipine maleate is prevented during long storage and reference dissolution rate is maintained, accordingly with excellent formulation-stability.

### ADVANTAGEOUS EFFECTS

The present invention provides a coated particle of amlodipine maleate for preventing a loss of amlodipine maleate during storage and distribution. The present invention also provides a pharmaceutical composition comprising the coated particle of amlodipine maleate for treating cardiovascular disease (CVD). The pharmaceutical composition has efficiencies on sufficient dissolution rate, stable bioavailability, formulation-stability equal to or more than one of amlodipine besylate during long storage, and/or formulation-stability enhanced regardless pH.

### BRIEF DESCRIPTION OF THE DRAWINGS

FIG. 1 represents a graph of blood concentration of formulations comprising a uncoated particle of amlodipine maleate (AM1) and a coated particle of amlodipine maleate (AM-8) prepared in Example 5.

### BEST MODE FOR CARRYING OUT THE INVENTION

The following examples are intended to describe the present invention in further detail and should not be constructed as limiting the scope of the present invention.

### Example 1

Amlodipine maleate (Manufacturer: UniChem) 320 g and talc 200 g was stirred in fluidized bed granulator, OPADRY-AMB® 700 g and talc 300 g was dispersed in water 10 kg and then the dispersed solution was sprayed into the granulator to form a coated amount of 170 weight percent over powder of amlodipine maleate. The resultant particle was thoroughly dried in the granulator and sieved with No. 35 sieve to form coated particles of amlodipine maleate.

### Example 2

Amlodipine maleate (Manufacturer: Kyongbo Pharm. Co., Ltd) 320 g and talc 200 g was stirred in fluidized bed granulator, OPADRY-AMB® 700 g and talc 300 g was dispersed in water 10 kg and then dispersed solution was sprayed into the granulator to form a coated amount of 170 weight percent over powder of amlodipine maleate. The resultant particle was thoroughly dried in the granulator and sieved with No. 35 sieve to form coated particles of amlodipine maleate.

### Example 3

Amlodipine maleate (Manufacturer: Kyongbo Pharm. Co., Ltd) 320 g and talc 200 g was stirred in fluidized bed granulator, OPADRY-OYC-7000A® 700 g and talc 300 g was dispersed in methylene chloride 3 kg and anhydrous ethanol 7 kg and then dispersed solution was sprayed into the granulator to form a coated amount of 170 weight percent over powder of amlodipine maleate. The resultant particle was thoroughly dried in the granulator and sieved with No. 35 sieve to form coated particles of amlodipine maleate.

### Example 4

Amlodipine maleate (Manufacturer: Kyongbo Pharm. Co., Ltd) 320 g and talc 200 g was stirred in fluidized bed granulator, sugar 700 g and talc 300 g was dispersed in water 10 kg and then dispersed solution was sprayed into the granulator to form a coated amount of 170 weight percent over powder of amlodipine maleate. The resultant particle was thoroughly dried in the granulator and sieved with No. 35 sieve to form coated particles of amlodipine maleate.

### Example 5

Amlodipine maleate (Manufacturer: Kyongbo Pharm. Co., Ltd) 320 g and talc 200 g was stirred in fluidized bed granulator, OPADRY-AMB® 700 g and Anhydrous dibasic calcium phosphate 600 g was dispersed in water 12 kg and then dispersed solution was sprayed into the granulator to form a coated amount of 223 weight percent over powder of amlodipine maleate. The resultant particle was thoroughly dried in the granulator and sieved with No. 35 sieve to form coated particles of amlodipine maleate.

<Preparation of Tablet>

After mixing the coated particles prepared in the above examples respectively in a ratio of the below table 1, tablets of 200 mg per each tablet are produced using rotary tablet press machine.

| | AM-1 | AM-2 | AM-3 | AM-4 | AM-5 |
|---|---|---|---|---|---|
| Amlodipine maleate | 6.4 g | 6.4 g | — | — | |
| (as amlodipine) | (5.0 g) | (5.0 g) | | | |
| Coated particle of example 1 | — | — | 28.1 g | — | — |
| (as amlodipine) | | | (5.0 g) | | |
| Coated particle of example 2 | — | — | — | 28.1 g | — |
| (as amlodipine) | | | | (5.0 g) | |
| Coated particle of example 3 | | | | | 28.1 g |
| (as amlodipine) | | | | | (5.0 g) |
| Anhydrous dibasic | 63.0 g | 63.0 g | 63.0 g | 63.0 g | 63.0 g |
| calcium phosphate | | | | | |
| Microcrystalline cellulose | 124.6 g | 122.6 g | 100.9 g | 100.9 g | 100.9 g |
| Sodium starch glyconate | 4.0 g | 6.0 g | 6.0 g | 6.0 g | 6.0 g |
| Magnesium stearate | 2.0 g | 2.0 g | 2.0 g | 2.0 g | 2.0 g |
| pH | 5.7 | 5.8 | 6.1 | 5.9 | 6.2 |

| | AM-6 | AM-7 | AM-8 | AM-9 | AM-10 |
|---|---|---|---|---|---|
| Amlodipine maleate | — | — | — | 6.4 g | — |
| | | | | (5.0 g) | |
| Coated particle of example 3 | — | — | — | — | 28.1 g |
| (as amlodipine) | | | | | (5.0 g) |
| Coated particle of example 4 | 28.1 g | — | — | — | — |
| (as amlodipine) | (5.0 g) | | | | |
| Coated particle of example 5 | — | 33.6 g | 33.6 g | — | — |
| (as amlodipine) | | (5.0 g) | (5.0 g) | | |
| Anhydrous dibasic | 63.0 g | 63.0 g | 63.0 g | 63.0 g | 63.0 g |
| calcium phosphate | | | | | |
| Microcrystalline cellulose | 100.9 g | 95.4 g | 92.9 g | 120.6 g | 98.9 g |
| Sodium starch glyconate | 6.0 g | 6.0 g | 6.0 g | 6.0 g | 6.0 g |
| Succinic acid | — | — | 2.5 g | 2.0 g | 2.0 g |
| Magnesium stearate | 2.0 g | 2.0 g | 2.0 g | 2.0 g | 2.0 g |
| pH | 6.3 | 6.0 | 4.9 | 4.7 | 4.8 |

In the above tables, AM represents amlodipine maleate.

### Experimental Example 1

### Test of Dissolution Rate and Content Loss for Formulation-Stability

An experiment of dissolution rate was carried out by dissolving respectively the above-prepared tablets and a tablet containing amlodipine besylate, has been sold (Novasc®, containing 5 mg as amlodipine), in 500 mL of 0.01 mol/L hydrochloric aqueous solution at a temperature of 37° C. for 30 minutes at 75 rpm. And an experiment of content loss, with respect to initial content, was performed by storage as opened at a relative humidity (RH) of 75 weight percent and 100 weight percent respectively for 3 months. The result is represent in the below table.

| | AM-1 | AM-2 | AM-3 | AM-4 | AM-5 |
|---|---|---|---|---|---|
| Dissolution rate | 89.5% | 91.1% | 92.6% | 91.3% | 86.5% |
| Content loss in a condition of | 4.6% | 13.8% | 0.2% | 0.1% | 1.3% |
| 40° C. and R.H. 75% after 3 months | | | | | |
| Content loss in a condition of | 11.5% | — | 5.6% | — | 6.1% |
| 50° C. and R.H. 100% after 3 months | | | | | |

| | AM-6 | AM-7 | AM-8 | AM-9 | AM-10 | Novasc ® |
|---|---|---|---|---|---|---|
| Dissolution rate | 84.6% | 90.6% | 91.0% | 91.0% | 92.0% | 88.5% |
| Content loss in a condition of | 1.0% | 0.0% | 1.0% | 14.2% | 1.9% | 1.1% |
| 40° C and R.H. 75% after 3 months | | | | | | |
| Content loss in a condition of | — | 4.9% | 5.8% | 35.2% | 6.8% | 7.0% |
| 50° C. and R.H. 100% after 3 months | | | | | | |

In the above table, AM represents amlodipine maleate.

The above table showed that formulation-stability of the tablet comprising the coated particles of amlodipine maleate, compared with one of Novasc®, was excellently enhanced regardless pH of tablets. However the tablets (AM-1 and AM-2), containing uncoated particles of amlodipine maleate, had low formulation-stability in a tablet pH range of 5.5 to 7.0 compared with one of Novasc®. Further the tablet (AM-9), containing uncoated particles of amlodipine maleate, out of the tablet pH range had rapidly content loss.

Accordingly, the pharmaceutical composition, comprising the coated particle of amlodipine maleate according to the present invention prevents content loss of amlodipine maleate during storage and circulation, and has excellent efficiencies on formulation-stability equal to or more than tablet containing amlodipine besylate and regardless pH.

### Experimental Example 2

### Bioequivalence Test

To confirm high bioavailibility of amlodipine maleate, bioequivalence test was carried out on tablets of the present invention and the conventional tablet.

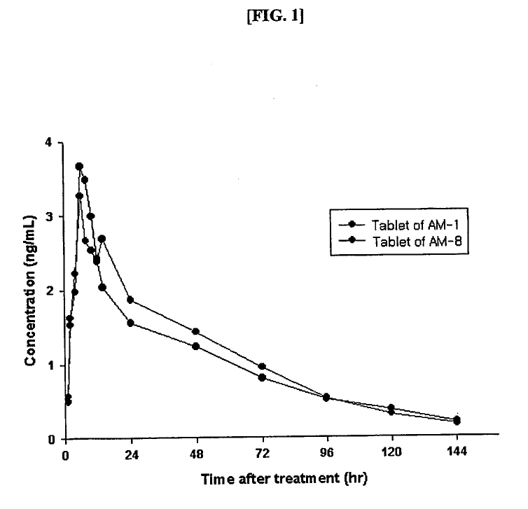

Into 12 men at an age of 19 to 55 dosed the tablet (AM-1) comprising amlodipine maleate and the tablet (AM-8) containing the coated particle of amlodipine maleate respectively, and after 1, 2, 4, 6, 8, 10, 12, 14, 24, 48, 72, 96, 120 and 144 hours, blood concentration of amlodipine was measured. The result referred to FIG. 1. And proper of pharmacokinetics was represented in the below table (Testing laboratory: As an medical center).

| Example | Tablet of AM-1 | Tablet fo AM-8 |
|---|---|---|
| Tmax (hr)* | 6.00 (2.00-14.00) | 8.00 (2.03-23.35) |
| Cmax (ng/mL) | 4.74 ± 2.42 | 4.68 ± 2.62 |
| AUC(0→∞) (ng · hr/mL) | 168.09 ± 94.12 | 158.97 ± 88.38 |
| T_{1/2β}(hr) | 35.28 ± 17.52 | 34.79 ± 20.29 |
| MRT (hr) | 43.25 ± 7.63 | 43.55 ± 8.39 |

*mean (range)

As showed in the above table, the pharmaceutical composition comprising the coated particle of amlodipine maleate has excellent efficiencies on property of pharmacokinetics equal to or more than conventional compositions containing amlodipine maleate according to testing, such as time that takes to blood maximum concentration of drug (Tmax), blood maximum concentration of drug (Cmax), bioavailibility (AUC→∞), half life (T_{1/2β}) and staying time of drug in blood.

Therefore the pharmaceutical composition of the present invention has excellent dissolution rate with respect to reference and stable bioavailibility equal to or more than the conventional pharmaceutical composition comprising uncoated particles of amlodipine maleate.

1. A coated particle of amlodipine maleate, comprising amlodipine maleate and pharmaceutically acceptable coating agent.

2. The coated particle of amlodipine maleate of claim 1, wherein the coating agent is one or a mixture selected from a water-soluble polymer group and a saccharine group

3. The coated particle of amlodipine maleate of claim 2, wherein the water-soluble polymer group comprises at least one selected from the group consisting of polyvinylpyrolidone, a cellulose group, pectine, galactomanan, gelatine, a polyethyleneglycol group, polymetacylate, a cyclodextrin group, carbomer and polyvinylalcohol.

4. The coated particle of amlodipine maleate of claim 3, wherein the cellulose group comprises celluloses selected from the group consisting of hydroxy propyl methylcellulose, hydroxypropylmethylcellulose pthalate, methylcellulose, carboxymethylcellulose sodium, hydroxyethylcellulose and hydroxypropylcellulose.

5. The coated particle of amlodipine maleate of claim 3, wherein the cyclodextrin group comprises cyclodextrin selected from the group consisting of betacyclodextrin, methylbetacyclodextrin and hydroxypropylbetacyclodextrin.

6. The coated particle of amlodipine maleate of claim 2, wherein the saccharide group comprises above one selected from the group consisting of sugar, sorbitol and mannitol.

7. The coated particle of amlodipine maleate of claim 1, wherein the coating agent is water-insoluble polymer by which coating is broken in 0.01 mol/L hydrochloric aqueous solution.

8. The coated particle of amlodipine maleate of claim 1, further comprising at least one selected from the group consisting of talc, light anhydrous silicic acid, silicon dioxide and anhydrous dibasic calcium phosphate.

9. A pharmaceutical composition for cardiovascular disease (CVD) comprising pharmaceutically acceptable carriers and the coated particle of amlodipine maleate represented claim 1 through claim 8 as active substance.

10. A method of preparing the coated particle of amlodipine maleate by coating a particle of amlodipine maleate with the coating agent in fluid bed granulator.

11. The method of claim 10, wherein the coating agent is one or a mixture of above two selected from light anhydrous silica acid and silicon dioxide.
